(19) United States
(12) Patent Application Publication
(10) Pub. No.: US 2025/0242172 A1
(43) Pub. Date: Jul. 31, 2025

(54) DEVICE AND METHOD FOR PROVIDING BLUE LIGHT THERAPY IN THE TREATMENT OF BRAIN DISORDERS

(71) Applicant: Mike Barnes, Dunnellon, FL (US)

(72) Inventor: Mike Barnes, Dunnellon, FL (US)

(21) Appl. No.: 19/040,683

(22) Filed: Jan. 29, 2025

Related U.S. Application Data
(60) Provisional application No. 63/738,896, filed on Dec. 26, 2024.
(60) Provisional application No. 63/630,396, filed on Jan. 31, 2024.

(51) Int. Cl. A61N 5/06

(52) U.S. Cl. CPC A61N 5/0622; A61N 2005/0626; A61N 2005/0632; A61N 2005/0647; A61N 2005/0652; A61N 2005/0663

(57) ABSTRACT

A blue light therapy device includes a domed shaped main body having a fixed array of optical emitters along the inside surface. Each of the optical emitters of the fixed array includes a blue light LED that applies optical emissions to the hippocampus and entorhinal cortex area of the recipient's brain in form of blue light between 400 nm and 470 nm to each side of the recipient's head at a location just above their ears. A second array of optical emitters are located on adjustable arms that are positionable within the nostrils of the recipient to apply optical emissions to the frontal and temporal lobes of the brain of the recipient. The device also includes a controller that is connected to the arrays, a user interface, an eye shield, and a chin strap.

### CROSS-REFERENCE TO RELATED APPLICATIONS

This application claims the benefit of U.S. Application Ser. No. 63/630,396 filed on Jan. 31, 2024, and U.S. Application Ser. No. 63/738,896 filed on Dec. 26, 2024, the contents of each of which are incorporated herein by reference.

### TECHNICAL FIELD

The present invention relates generally to light producing devices, and more particularly to a device and method for providing blue light therapy.

### BACKGROUND

The statements in this section merely provide background information related to the present disclosure and may not constitute prior art.

Alzheimer's disease is a brain disorder that gradually destroys a person's memory and ability to perform routine tasks; whereas Parkinson's disease is a chronic brain disorder that causes movement problems, stiffness, and balance issues. Both diseases are progressive, diseases that predominantly affect older adults. Although the exact causes and criteria for developing Alzheimer's and Parkinson's are not fully known, recent studies have suggested that Porphyromonas gingivalis may play a role in the development of these debilitating diseases.

Porphyromonas gingivalis is a bacterium primarily linked to periodontal disease (gum disease), but emerging research strongly suggests that it may have broader neurological implications. Recent studies have suggested that Porphyromonas gingivalis can enter the bloodstream and possibly travel to the brain, where it plays a role in the development of neurodegenerative diseases such as Alzheimer's disease and Parkinson's disease, for example.

Patients with Alzheimer's disease have a high accumulation of beta-amyloid plaques in the brain. These plaques disrupt communication between brain cells and lead to inflammation and neuronal damage. Porphyromonas gingivalis produces gingipains, which are enzymes that break down proteins and can promote the aggregation of beta-amyloid. This aggregation can accelerate the formation of amyloid plaques, contributing to Alzheimer's disease pathology. Additionally, Porphyromonas gingivalis is believed to activate the brain's immune response, leading to neuroinflammation that further exacerbates the disease.

Parkinson's disease also involves the accumulation of alpha-synuclein protein into Lewy bodies inside neurons. There is emerging evidence suggesting that Porphyromonas gingivalis might play a role in this aggregation by triggering inflammation in the brain, which could encourage alpha-synuclein to misfold and form clumps, contributing to the neurodegenerative process. Indeed, some research suggests that Porphyromonas gingivalis may also influence the gut-brain axis, which could be relevant for Parkinson's, as alpha-synuclein aggregation has been shown to start in the gut and spread to the brain.

Although there have been a number of surgical and pharmaceutical approaches to slow or cure the onset and development of Alzheimer's and Parkinson's, none of these approaches have achieved ultimate success thus far. Moreover, each of these treatments are invasive to the patient and result in side effects that can be painful or uncomfortable.

Accordingly, it would be beneficial to provide a device and method for treating some of the underlying causes of these and other brain disorders that utilizes non-invasive optical emitters, so as eliminate the drawbacks of the treatments noted above.

### SUMMARY OF THE INVENTION

The present invention is directed to a blue light therapy device. One embodiment of the present invention can include a domed-shaped main body having an inside surface and an outside surface. A plurality of optical emitters can be positioned along the inside surface of the main body. Each of the plurality of optical emitters can include blue light LED's and can be grouped into two arrays. Each of the two arrays can be positioned along the sides of the main body and apply optical emissions at a target wavelength to the first target area of a recipient wearing the main body on their head.

In one embodiment, the first target area is the hippocampus and entorhinal cortex area of the recipient's brain, and the target output can be blue light in the range of between 400 nm and 470 nm to each side of the recipient's head at a location just above their ears.

In one embodiment, the device can include a second array of optical emitters that are positioned along adjustable arms. The second array of optical emitters can be positioned within the nostrils of the recipient to apply optical emissions at the target wavelength to a second target area. The second target area can comprise the frontal and temporal lobes of the brain of the recipient.

In one embodiment, the device includes a controller that is connected to the arrays and to a user interface for controlling the operation of the optical emitters. In one embodiment, a shield is removably positioned along the main body for protecting the eyes of the recipient, and a chin strap is provided for securing the main body to the recipient's head.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a back perspective view of a blue light therapy device in accordance with one embodiment of the invention.

FIG. 2 is a bottom perspective view of the blue light therapy device in accordance with one embodiment of the invention.

FIG. 3 a simplified block diagram of the system controller of the device, in accordance with one embodiment of the invention.

FIG. 4 is a front view of the device in operation, in accordance with one embodiment of the invention.

FIG. 5 is another front view of the device in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “optical emitter” can include any form of light producing and/or light emitting device having any number of different colors across the color spectrum. Several non-limiting examples can include Light Emitting Diodes (LED's), light emitting capacitors, and/or super-luminous light emitting diodes, for example, that are capable of individually and/or jointly creating optical emissions of light/radiation at a target wavelength.

As described herein, a “target wavelength” and an “optical output” shall be used interchangeably to describe the measurable light/radiation output of one or more optical emitters, and/or the below described array that is sufficient to apply blue light in the range of between 400 nm-470 nm to the target area(s) of the patient.

Through extensive research and testing, the above noted inventor has developed a device and treatment for targeting and reducing Porphyromonas gingivalis in human patients through exposure to photonic energy that is applied by optical emitters at a target wavelength. In this regard, the below described device 10 can function to kill Porphyromonas gingivalis located in the head of a user by targeting the veins and arteries leading to and from the brain.

Because the hippocampus and entorhinal cortex area of a patients brain are the areas most commonly affected by early onset Alzheimer's disease, the device can concentrate a plurality of fixed optical emitters to illuminate the patients head at a location just above (e.g., between 0.5 inches and 2 inches) each ear, so as to target this area of the brain as a first target area.

Additionally, because the frontal and temporal lobes are the portion of the brain where high memory functions reside, the device can concentrate a pair of adjustable optical emitters to illuminate these portions. Other than the eyes, the trigeminal pathway provides a direct pathway to the frontal lobe of the brain without going through a significant amount of bone, as such, the adjustable optical emitters can be positioned by a user within the nose of a patient to target this area of the brain as a second target area. Of course, the device is not limited to treatment of these particular target areas, as additional optical emitters can be positioned at other locations for illuminating other portions of a patient's body.

In either instance, the device can illuminate the epidermis, subcutaneous tissue, veins, and arteries of a patient by applying light/radiation from an array of optical emitters at a target wavelength. In the preferred embodiment, the target wavelength is in the range of between 400-470 nm, which is specifically chosen to kill Porphyromonas gingivalis in the target area. Such a feature acting to reducing the amount of Porphyromonas gingivalis reaching the brain with the goal of reducing or eliminating the onset of several types of brain diseases.

Because human skin, the underlying tissue, and bone reflect and/or absorb light, it is necessary that the measurable light/radiation output leaving the array be sufficient to make contact with the veins and arteries in the target area. Because blue light penetrates deeper than other colors such as red or orange, it can travel further through the body before being absorbed. Nonetheless, it is preferred that the array be positioned directly against the skin of the patient, in order to penetrate the body and deliver the maximum treatment to the target area(s).

Although described predominantly for the treatment of brain disorders, tests utilizing the inventive device have shown several promising potential supplemental benefits to patients such as: promoting cellular healing, enhancing mitochondrial functions, stimulating blood flow, and improving overall brain functionality, for example.

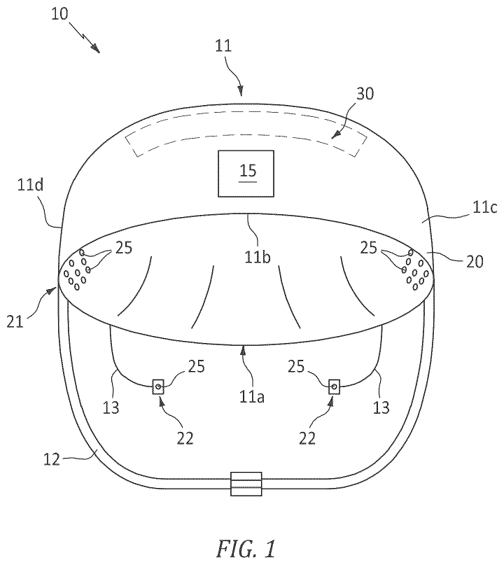

Turning now to the drawings, where identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

FIGS. 1-4 illustrates one embodiment of a device 10 for providing blue light therapy in the treatment of brain disorders that is useful for understanding the inventive concepts disclosed herein. As shown, the device 10 can include a main body 11 having two arrays of fixed optical emitters 20 and 21, and an adjustable array of optical emitters 23 which can operate alone or together to produce photonic energy in the form of light/radiation at a target wavelength when instructed by a controller 30.

As shown best at FIG. 1, one embodiment, the main body 11 can take the form of a hat or helmet that is designed to be positioned onto the head of a user. In this regard, the main body 11 can include a generally dome-shaped member having a front end 11a, a back end 11b, and a pair of sides 11c and 11d. The main body can be constructed from any number of durable lightweight materials such as various plastics, for example, however other materials are also contemplated.

In one embodiment, an optional chin strap 12 can extend out from the bottom portion of the main body for securing the device onto the head of a user. In various embodiments, the chin strap may include a single elastomeric strap or may include two straps that are joined together via a buckle or other device.

In one embodiment, a pair of adjustable array arms 13 can extend outward from the front end of the helmet. Each of the arms 13 can ideally be constructed from a malleable material such as plastic or bend and stay tubing, for example, which can be bent, twisted or otherwise manipulated into any number of different shapes, and that will retain that shape indefinitely.

In one embodiment, a user interface 15 can be provided along the main body 11. The user interface can be communicatively linked to the system controller 30 and each of the arrays 20 and 25 described below. In the preferred embodiment, the user interface device can include a touchscreen display having a Graphic User Interface (GUI) for providing two-way communication with a user.

To this end, the interface can allow the user to instruct the device to perform functions such as transitioning the arrays an ON and OFF operating state, selectively activating the arrays individually and/or individual optical emitters, adjusting the output of the emitters and for selecting the durational output of the same. Of course, the user interface can also include, comprise, or consist of any number of buttons, switches, or other such devices for performing this functionality.

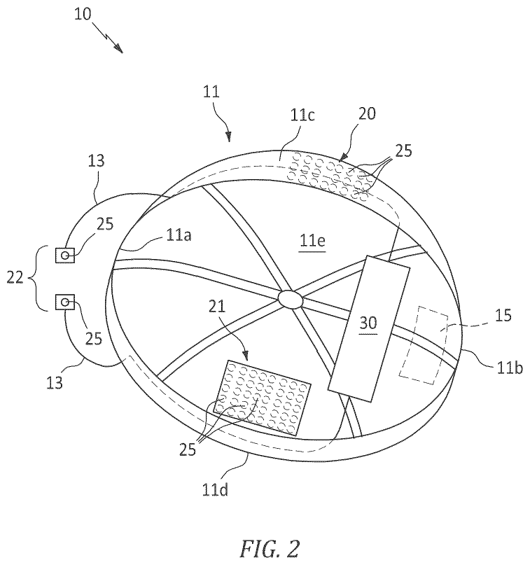

As shown best at FIG. 2, a pair of fixed optical arrays 20 and 21 can be positioned along the inside surface 11e of the main body. The arrays can include a plurality of individual optical emitters 25 that function to illuminate the skin, skull, tissue, veins, and arteries of a patient's head that surround and interact with the patient's brain.

In the preferred embodiment, the optical emitters 25 in each of the arrays 20 and 21 can include 24 individual 3 W-10 W blue LED's. As shown, each of the arrays of optical emitters 20 and 21 can be positioned along the sides 11c and 11d, respectively, of the main body 11 in order to specifically and cumulatively engage the first target area of the patient wearing the main body to provide treatment to the patient's hippocampus and entorhinal cortex area.

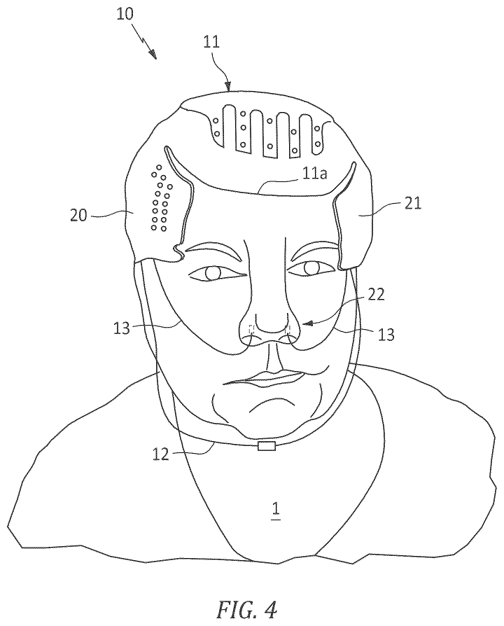

In one embodiment, an adjustable array 22 of optical emitters can be positioned along the distal ends of the adjustable array arms 13. In the preferred embodiment, the optical emitters 25 of the adjustable array 22 can include between 2 and 4 individual 3 W-10 W blue LED's. As shown below at FIG. 4, the arms 13 allow a user wearing the device to position the optical emitters of the adjustable array 22 into their nostrils to provide treatment to the second target area comprising the patient's trigeminal pathway and frontal lobe.

In either instance, each of the LED's 25 of the above noted arrays 20, 21 and 22 can be connected to the LED driver associated with the component interface unit 33 of the below described controller 30 having a preferred electrical output of 200 ma to 4A at a frequency of 25 hz-35 hz. When so provided, each of the LED's will output optical emissions to the respective target areas at the target wavelength that are between about 400-470 nm.

Although not specifically illustrated, each of the LED arrays and/or the controller components can incorporate any number of heat dissipating elements such as active fan-based ventilation or more preferably heat syncs to reduce the buildup of heat experienced by the patient during operation of the device.

Moreover, although described above as including a specific shape, size, construction material, type and/or number of optical emitters, this is for illustrative purposes only, as those of skill in the art will recognize that many different combinations, numbers and types of optical emitters and/or main body shapes and construction materials can also be utilized to achieve the desired optical output described herein. To this end, other embodiments are contemplated wherein additional fixed arrays are positioned within the main body to directly target additional areas of the brain.

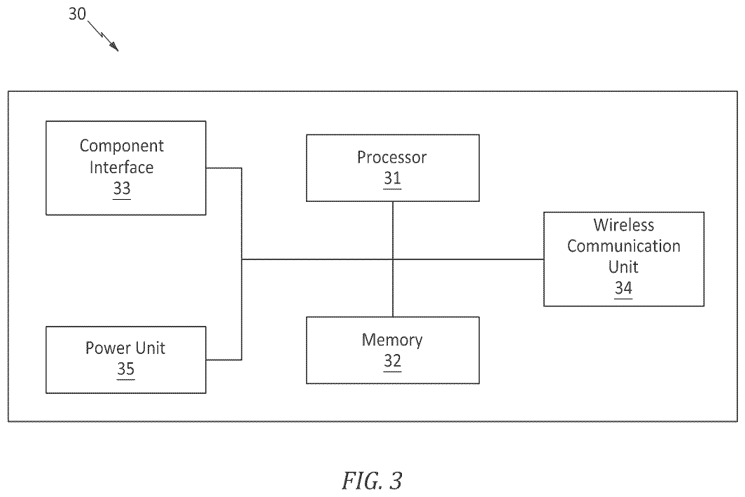

FIG. 3 is a simplistic block diagram illustrating one embodiment of the controller 30 that includes a processing unit 31 that is conventionally connected to an internal memory 32, a component interface unit 33, a wireless communication unit 34, and/or a power unit 35.

Although illustrated as separate elements, those of skill in the art will recognize that one or more system components may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. The bulk storage device can contain any number of different programs that permit the processor to perform the functionality described herein and can also receive and store the exercise information for each user.

The component interface unit 33 can function to provide a communicative link between the processing unit 31, the optical emitters 25 of the arrays 20, 21 and 22, and the user interface device 15. In this regard, the component interface unit can include any number of different components such as an LED driver, one or more PIC microcontrollers, standard bus, internal bus, connection cables, wireless receiver and/or associated hardware such as USB cables and connectors, and other such hardware capable of linking the various components. Of course, any other means for providing the two-way communication between the system components can also be utilized herein.

The communication unit 34 can include any number of components capable of sending and/or receiving electronic signals with another device, either directly or over a network. In the preferred embodiment, the communication unit 34 can include a Bluetooth transceiver for communicating directly with an application running on a user's smartphone or tablet device. The App includes functionality for duplicating the features of the user interface to allow a user to control the operation of the device 10 via the app. Of course, any number of other types of wireless transceivers may also be utilized herein.

The power unit 35 can include any number of different components capable of providing the necessary power requirements to each element of the device. In the preferred embodiment, the power source can include or comprise one or more batteries that are positioned within the main body. The batteries may be removeable via an access panel or rechargeable via a cable or wireless charging system.

FIG. 4 illustrates one embodiment of the device 10 in operation. As shown, a user 1 can position the main body 11 onto their head and tighten the chin strap 12 to position the fixed arrays 20 and 21 over the first target area to apply optical emissions at the target wavelength to treat the first target area.

Additionally, the user can manipulate the adjustable arms 13 to position the optical emitters of the second array 22 within their nostrils. When so positioned, the optical emitters of the second array will apply their optical emissions at the target wavelength to treat the second target area.

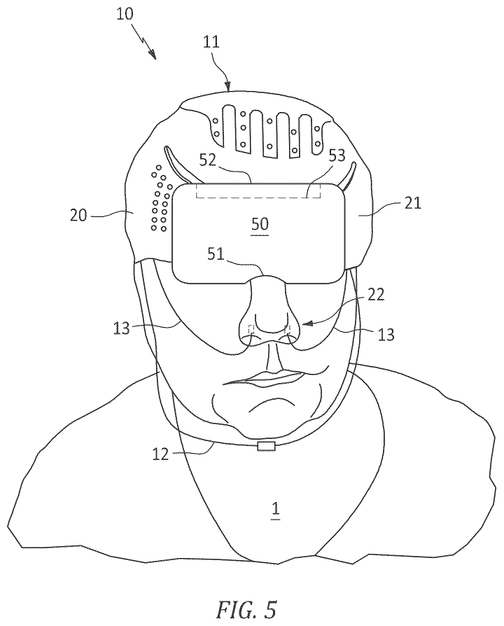

FIG. 5 illustrates one embodiment of the device 10 that further includes an eye shield 50. As shown, the eye shield can include an elongated member having a curvilinear bottom end 51 and a top end 52 that is removably connected to the front of the main body via a connector 53 such as strips of hook and loop material or a clip, for example. The shield 50 can ideally be constructed from a lightweight and opaque material such as plastic, for example, and can function to prevent the optical emissions from the adjustable array 22 from being directed into the eyes of a user during operation of the device.

Accordingly, the above noted device functions to selectively apply optical emissions to specific target areas of a patient in a manner that is designed to kill Porphyromonas gingivalis and reduce the onset of debilitating brain disorders.

As to a further description of the manner and use of the present invention, the same should be apparent from the above description. Accordingly, no further discussion relating to the manner of usage and operation will be provided.

As described herein, one or more elements of the device 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A blue light therapy device, comprising:
a main body having an inside surface and an outside surface;
at least one array of optical emitters that is positioned along the main body, said at least one array being configured to be oriented toward at least one target area of a recipient to be treated;
a user interface that is positioned along the outside surface of the main body; and
a controller that is positioned along the main body, said controller being in communication with each of the user interface and the at least one array of optical emitters,
wherein the at least one array of optical emitters is configured to selectively produce optical emissions at a target wavelength to the at least one target area.

2. The device of claim 1, wherein the main body includes a dome-shape and is configured to be positioned about a head of the recipient to be treated.

3. The device of claim 2, wherein the at least one array of optical emitters comprises:
a first array of optical emitters that are fixedly positioned along the inside surface of the main body, and a second array of optical emitters that are fixedly positioned along the inside surface of the main body.

4. The device of claim 3, wherein the first array of optical emitters is positioned along a first side of the main body, and the second array of optical emitters is positioned along a second side of the main body.

5. The device of claim 4, wherein the first array of optical emitters is configured to engage the head of the recipient at a location above a right ear, and
the second array of optical emitters is configured to engage the head of the recipient at a location above a left ear.

6. The device of claim 5, wherein the first target area comprises a hippocampus and an entorhinal cortex of a brain of the recipient to be treated.

7. The device of claim 1, wherein each of the optical emitters of the at least one array of optical emitters comprise a light emitting diode.

8. The device of claim 1, wherein the target wavelength comprises blue light in the range of between about 400 nm to 470 nm.
